(19) United States
(12) Patent Application Publication
(10) Pub. No.: US 2019/0217077 A1
(43) Pub. Date: Jul. 18, 2019

(54) DEVICES AND METHODS TO MAINTAIN CLEAN SURFACES ON MEDICAL CONNECTORS

(71) Applicant: Daniel M. CHAMBERS, Solana Beach, CA (US)

(72) Inventor: Daniel M. CHAMBERS, Solana Beach, CA (US)

(21) Appl. No.: 16/247,527

(22) Filed: Jan. 14, 2019

Related U.S. Application Data
(60) Provisional application No. 62/617,170, filed on Jan. 12, 2018.

(51) Int. Cl. A61M 39/22; A61M 39/18; A61M 39/10

(52) U.S. Cl. CPC A61M 39/22; A61M 39/02; A61M 39/1011; A61M 39/18

(57) ABSTRACT

Described are disposable, single-use fluid access port cover devices for medical connectors such as luer access valves, including needless, valved connectors (i.e., needless valves). Such device typically include a plastic backing element that preferably includes a device removal tab and a recess adapted to receive a port cover matrix having an antimicrobial agent dispersed or disposed therein.

This application claims the benefit of and priority to U.S. provisional patent application No. 62/617,170, filed on 12 Jan. 2019 and having the same title (attorney docket number IVO-0015-PV), the contents of which are hereby incorporated by reference in their entirety for any and all purposes

### TECHNICAL FIELD OF THE INVENTION

This invention is directed to devices that maintain clean surfaces on medical connectors, particularly luer access devices such as needleless, valved connectors, and methods for using such articles.

### BACKGROUND OF THE INVENTION

### 1. Introduction

The following description includes information that may be useful in understanding the present invention. It is not an admission that any such information is prior art, or relevant, to the presently claimed inventions, or that any publication specifically or implicitly referenced is prior art.

### 2. Background

In the medical field, and in particular the area of infusion of fluids or aspiration of fluids to or from a patient, there remains a need to prevent the transmission of pathogens into or onto a patient from a potentially contaminated surface of a medical device such as a luer access device, for example, a needleless, valved connector. Pathogens include microorganisms such as bacteria and viruses, the transmission of which into a patient may result in an infection that could be life threatening. Specific to healthcare settings, the term “nosocomial infection” describes those infections that originate from or occur in a hospital or hospital-like setting. In the U.S., nosocomial infections are estimated to occur in at least 5% of all acute care hospitalizations. The estimated incidence is more than two million cases per year, resulting in significant morbidity, mortality, and an expense. Indeed, nosocomial infections are estimated to more than double the mortality and morbidity risks of any admitted patient, and likely result in about 90,000 deaths a year in the United States. Common sites for such transmissions are found on such medical connectors as luer access devices, vials, needleless (or needle free) valves, and the injection ports of valves, tubing, and catheters. The incidence of such infections in patients is increasing, and infection control practitioners (ICP's) often cite improper cleansing of sites as a major source of these infections.

As described above, exposure to pathogens and infectious agents (e.g., pathogenic bacteria, viruses, fungi, etc.) in medical settings is a matter of serious concern. One route of exposure to such agents is the opening made in skin provided by the bore of needle, cannula, or other similar device used to provide access to a patient's vasculature. It is known that patients whose skin has been compromised in this way are at increased risk for developing serious blood stream infections. In the United States alone, approximately 300,000 blood stream infections per year result from the installation and use of peripheral intravenous catheters (PIVC), and more than 80,000 blood stream infections are associated with the use central venous catheters (CVC) and peripherally inserted central catheters (PICC). All told, in the U.S. approximately 20,000 patients die annually from hospital-acquired infections that result from PIVC, CVC, and PICC use. Costs associated with the care and treatment of patients that develop infections due to PIVC, CVC, and PICC use exceed $2.7 billion annually.

In hospital settings today, occupational health and safety regulations designed reduce the risk to health care workers from needle prick and similar injuries have resulted in the deployment of needleless medical valves whenever possible. Currently, more than 500 million needleless valves are used annually in hospitals throughout the U.S. Needleless valves are used primarily in conjunction with PIVC, CVC, and PICC devices, which may contain from as few as one to as many as 3, 4, 5, or more needleless valves. FIG. 1 illustrates an example of a representative needleless medical valve in use today.

The widespread use of needleless valves in acute medicine has contributed to a marked increase in the incidence of hospital-acquired infections (HAIs), particularly blood stream infections, which are also known as catheter-related blood stream infection (CRBSIs). To reduce the risk of infection from a contaminated needleless valve, standard practice today requires that a nurse or other health care worker clean the surfaces of a needleless valve by rubbing it with a sterile alcohol swab or wipe immediately prior to making a connection to the valve, for example, by attaching a syringe to the valve to deliver a medication via a PIVC, CVC, or PICC already connected to a patient. Given the magnitude of the mortality and morbidity associated with HAIs and the large number of CRBSIs that result from PIVC, CVC, and PICC use, a long-recognized yet significant unmet need exists for articles or devices that can be used to reduce or eliminate the risk of initiating an HAI merely by accessing a patient's vasculature through a needleless valve component of a PIVC, CVC, or PICC inserted into a blood vessel of a patient.

Traditionally, cleaning or cleansing a potentially contaminated surface involved a protocol of alcohol swabbing prior to making the necessary connections to the site. Alcohol swabs are typically a small pad of cotton gauze or other absorbent material soaked in a 70% isopropyl alcohol (IPA) or ethanol (EtOH) solution, packed individually in a foil package to prevent evaporation of the alcohol solution from the swab prior to use. Properly used, the package is opened at or near the site to be swabbed. With gloved hands, the swab is removed by a nurse or other healthcare provider and wiped across the top and side surfaces of the sites to be connected. After use, the swab and foil package are discarded and the site allowed to dry, usually 20-30 seconds, immediately prior to making the desired device connection(s). This “drying” period is important because, as the alcohol dries, it breaks open the cellular walls of microorganisms, thereby killing them.

Unfortunately, because of increased duties and responsibilities, shrinking nursing staffs, and inadequate training, alcohol swabbing is often not performed or is poorly executed. A poorly swabbed site can carry microorganisms that, if allowed to enter a patient's body, can cause serious infection. In addition, supervisory oversight is nearly impossible, because unless a supervisor actually observes swabbing as it is performed, the supervisor cannot know whether or not it was done properly or performed at all. Further, without at least a sufficient microscopic examination for microbial residue (e.g., biofilm), there may be no evidence of an alcohol swab being performed.

Thus, a significant need still exists for devices and techniques to maintain cleanliness of access sites on medical connectors in the field, particularly in acute care settings.

### 3. Definitions

Before describing the instant invention in detail, several terms used in the context of the present invention will be defined. In addition to these terms, others are defined elsewhere in the specification, as necessary. Unless otherwise expressly defined herein, terms of art used in this specification will have their art-recognized meanings.

As used herein, the singular forms “a”, “an”, and “the” include plural references unless the context clearly dictates otherwise.

As used herein, the term “about” refers to approximately a +/−10% variation from the stated value. It is to be understood that such a variation is always included in any given value provided herein, whether or not it is specifically referred to.

The term “biofilm” refers to a matrix of microorganisms and extracellular material attached to a surface that enables the microorganisms, typically bacteria and/or fungi, to adhere to a surface and remain viable.

A “patentable” composition, process, machine, or article of manufacture according to the invention means that the subject matter at issue satisfies all statutory requirements for patentability at the time the analysis is performed. For example, with regard to novelty, non-obviousness, or the like, if later investigation reveals that one or more claims encompass one or more embodiments that would negate novelty, non-obviousness, etc., the claim(s), being limited by definition to “patentable” embodiments, specifically excludes the unpatentable embodiment(s). Also, the claims appended hereto are to be interpreted both to provide the broadest reasonable scope, as well as to preserve their validity. Furthermore, if one or more of the statutory requirements for patentability are amended or if the standards change for assessing whether a particular statutory requirement for patentability is satisfied from the time this application is filed or issues as a patent to a time the validity of one or more of the appended claims is questioned, the claims are to be interpreted in a way that (1) preserves their validity and (2) provides the broadest reasonable interpretation under the circumstances.

A “plurality” means more than one.

“Single-use” (or “single purpose”) refers to an article or device suitable for one use or purpose only, as distinguished from “dual” or “multiple” use or purpose devices. Thus, in the context of the invention, a “single-use” fluid access port cover is one that is useful for covering, for example, the fluid access port of a needleless medical valve in order to prevent its contamination while exposed to the environment in an acute care patient setting; once the fluid access port cover is removed from the fluid access port, the cover is discarded (i.e., not used again to cover the same or different fluid access port).

### DESCRIPTION OF THE INVENTION

The object of the invention is to address these long-standing but still unmet needs. This invention addresses these needs by providing patentable, single-use cover devices or articles that can be used to effectively and efficiently cover otherwise exposed access sites of medical connectors such as luer access devices, e.g., needleless medical valves, particularly the accessible surface(s) of the valve stems of needleless valves, as such surfaces form part of the fluid communication pathway between an external-delivered fluid (e.g., IV fluids blood, plasma, medicine, etc.) and a patient's blood stream and may become contaminated with pathogens or infectious agents (e.g., bacteria, fungi, and viruses) in the environment, particularly since such connectors are often exposed to the environment when installed on patients in hospitals and acute care settings for up to 7 days.

Thus, in one aspect, the invention provides disposable, single-use fluid access port cover devices for medical connectors such as luer access valves, including needless, valved connectors (i.e., needless valves). In general, such access port cover devices include a backing element and a port cover matrix (preferably made of a compressible, even more preferably, absorbent, material). If the port cover matrix lacks adhesive characteristics that allow it to secure the device to the access port, the device will also include an adhesive layer to allow the device to be adhered to one or more surfaces of a medical connector so as to cover the connector's access port, thereby minimizing the access port's exposure to the environment. Preferably, the device of the invention also includes one or more antimicrobial agents disposed on or dispersed in a layer (e.g., the port cover matrix) that contacts the access port surface when the device is adhered to a medical connector. In some embodiments, the device also includes one or more removal tabs so as to facilitate removal of the port cover device when desired and thus present the access port for use.

The fluid access port cover devices of the invention also preferably include one or more release liners that cover the device's adhesive element(s), which liner(s) is(are) removed (e.g., by peeling them away) just prior to or during the process of affixing the device to a medical connector to cover the connector's access port to be covered and thus limit or prevent contamination of the access port's surface when or while it is exposed to the environment, particularly the environment in an acute care setting (e.g., a hospital room, a surgical theater, a hospital emergency room, an intensive care unit, etc.).

The port cover matrix, for example, an antimicrobial silicone adhesive (e.g., such an adhesive containing about 3% chlorhexidine and about 0.5% w/w silver salts), an open-cell foam, a woven or non-woven matrix, etc. is secured to a first surface of the backing element and is configured to contact and cover the surface of a fluid access port of a medical connector that, in the absence of a cap or cover, would otherwise be exposed to the environment. In some embodiments, particularly those wherein the port cover matrix is compressible and preferably absorbent, the port cover matrix attached to the backing element is compressed when the port cover is adhered to the area surrounding the access port of a medical connector.

In some embodiments, the antimicrobial agent(s) is(are) dispersed in the port cover matrix (particularly when the matrix is absorbent and suitable for use with a liquid disinfecting agent) at the time the device is manufactured, while in other embodiments, the antimicrobial agent(s) is(are) dispersed into the matrix just prior to the matrix coming into contact with the access port of a medical connector, for example, by rupturing a reservoir included in the port cover in order to saturate the port cover matrix with a disinfecting solution prior to it being adhered to a medical connector. Such a reservoir can be disposed, for example, between the backing element and port cover matrix. Preferred antimicrobial agents include disinfecting solutions such as 70% isopropyl alcohol or antibacterial agents and/or antifungal agents, for example, chlorhexidine, triclosan, benzalkonium chloride, ciprofloxacin, gentamicin, a silver agent, or combinations thereof. Representative examples of a silver agent include a silver salt, such as silver phosphate, silver citrate, silver lactate, silver acetate, silver benzoate, silver chloride, silver carbonate, silver iodide, silver iodate, silver nitrate, silver laurate, silver sulfadiazine, silver palmitate, and combinations thereof. In some embodiments, the antimicrobial agent is encapsulated and/or associated with a pharmaceutical carrier, such as liposomes, micelles, microcapsules, microspheres, nanospheres, or combinations thereof. In some embodiments, the antimicrobial agent further comprises a stabilizer, such as pyrrolidone carboxylic acid (PCA), Brilliant Green, Crystal Violet, or combinations thereof.

The port cover matrix is preferably sized to cover the exposed surface area of the valve portion of a medical connector, and the matrix is attached to the backing element. In some embodiments, the port cover matrix is positioned in the center of the backing element or housing such that the matrix portion is bounded about its outer periphery by an adherence zone. In some embodiments, the adherence zone is part of the port cover matrix, preferably a region that comprises the outer region of the matrix. In other embodiments, the adherence zone (or valve engagement region) is a portion of the backing element configured to associate with and removably engage the upper, outer region of the plastic body of the medical connector to which the access port cover is attached. In some preferred embodiments the adherence zone includes an adhesive, preferably a biocompatible adhesive, that can securely yet removably adhere the port cover to the surface(s) surrounding the access port of the medical connector to be covered. In preferred embodiments, the housing includes a recess adapted to receive the port cover matrix, which preferably is adhered with a suitable adhesive to the surface of the recess facing the matrix. Alternatively, or in addition, mechanical structures can be provided to engage and retain (or assist in engaging and retaining) the port cover matrix in the housing, particularly the recess in the matrix adapted for such purpose. The backing element can be made from any suitable material, preferably a biocompatible plastic, using any suitable process (e.g., injection molding, 3D printing, etc.).

In preferred embodiments, the access port covers of the invention include at least one removal tab to facilitate the cover's removal from a medical connector to expose a preferably clean fluid access port. The removal tab may be part of the backing element, or it may be a distinct element that is attached to the backing element during manufacture of the cover.

The access port covers of the invention also may include one or more release liners to protect the port cover matrix and adhesive in the adherence zone until just prior to the access port cover's use. At that point, a user, typically a healthcare worker, removes the access port cover from its (preferably sterile) packaging and, after cleansing the access port and preferably allowing it to dry, removes the release liner(s) and then immediately places the cover over the access port to be covered.

In those embodiments where the access port cover is sterilized during manufacture, release liners are preferably applied prior to sterilization. Access port covers according to the invention can be packaged individually or in two or more units. The packaged device(s) are then typically packaged in, for example, a labeled box that contains 20-500 or packaged units. Sterilization, if desired, can be performed by any suitable method, form example, e-beam, gamma radiation, or exposure to ethylene oxide gas. As those in the art will appreciate, the sterilization process employed (if any) will depend on various factors, including the chemical makeup of the various portions of the device (e.g., the plastic used to form the port cover, the port cover matrix, the antimicrobial agent(s) included in the port cover matrix, the packaging used for the device, etc.)

A related aspect of the invention concerns kits that include a packaged access port cover according to the invention and instructions for use of the port cover. Another related aspect of the invention concerns an access port cover removably attached to a needleless medical connector so as to cover the connector's valve surface. Yet another related aspect of the invention concerns methods of preventing or reducing microbial contamination of a valve surface of a needleless medical connector by attaching an access port cover to the valve portion of the connector to prevent exposure of that valve surface to an environment harboring contaminating microorganisms. Such methods of reducing contamination of a valve surface of a needleless medical connector typically involve removably attaching an access port cover of the invention to a needleless medical connector having a fluid valve such that the surface of the valve that opens contacts the port cover matrix of the access port cover.

Other aspects of the invention concern methods of covering medical connectors using an access port cover according to the invention.

Features and advantages of the invention will be apparent from the following detailed description, and appended claims.

### DESCRIPTION OF THE DRAWINGS AND REPRESENTATIVE EMBODIMENTS

These and other aspects will now be described in detail with reference to the following drawings. Unless otherwise indicated, it is understood that the drawings are not to scale, as they are intended merely to facilitate understanding of the invention as opposed to specific dimensions, etc. In the drawings, like numbers in two or more drawings represent like elements. In the figures, similar symbols typically identify similar components, unless context dictates otherwise. The illustrative embodiments described herein are not meant to be limiting. Other embodiments may be utilized, and other changes may be made, without departing from the spirit or scope of the subject matter presented here.

FIG. 1 shows two cross-sectional drawings (FIGS. 1A and 1B) of a representative diagrammatic illustration of some of the major components of a conventional needleless medical connector (1). In FIG. 1A, the connector's valve portion (3) is shown in a closed position. FIG. 1B shows the valve (3) in an open position, with the fluid path indicated by arrows. An adherence zone (7) is depicted on the outer surface of the valve body (5), preferably above the male threaded region (8), near the valve opening (9).

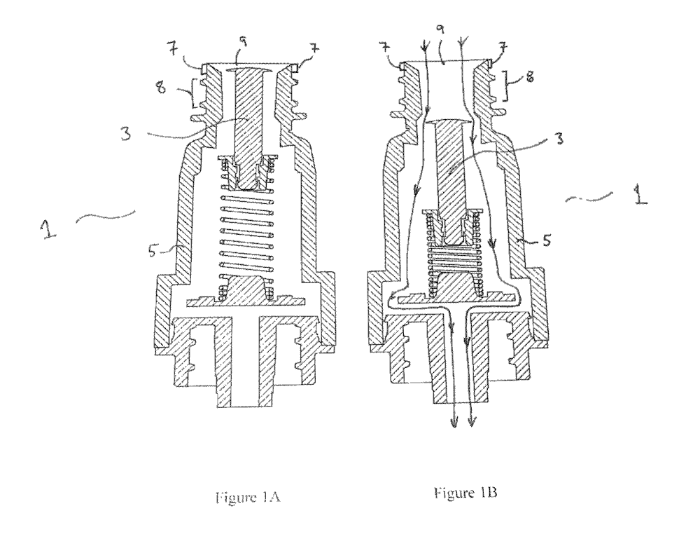

FIG. 2 shows two drawings (FIGS. 2A and 2B) of a representative single-use luer access access port cover of the invention (20). FIG. 2A shows the port cover housing (21) or backing element, the cover's removal tab ((22) here, a removal tab capable of being moved between a “closed” and “open” position, with the “open” position being used for removal of the port cover from a medical connector such as a luer access device), a port cover matrix (24) that contains the antimicrobial formulation (not shown)(e.g., isopropyl alcohol, a mixture of chlorhexidine and silver salts, etc.) disposed in a recess (not visible in this view) in the housing (21), and a partially removed seal ((26) or release liner). Also visible on the side wall of the recess is an adherence or engagement zone (27) adapted to allow the port cover device to be removably attached to the region just above the threaded valve region (2) of the medical connector (1). FIG. 2B shows a side view of a representative access port cover (20) as shown in FIG. 2A attached to a needleless medical connector (2) so as to cover the valve portion (not shown) of valve (which otherwise would be exposed to the environment), and also shows the cover's removal tab (22) in an “open” position to facilitate removal of the cover from the needleless medical connector.

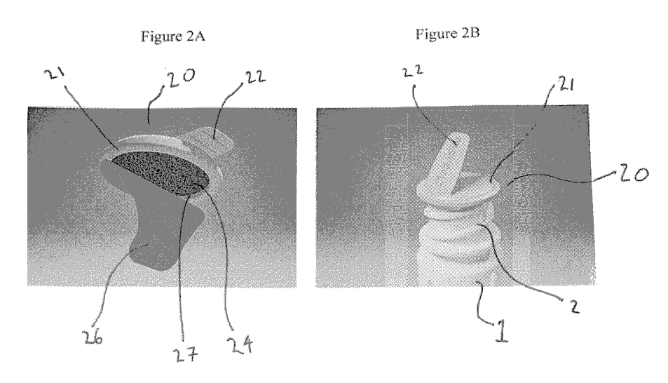

Unless the context clearly requires otherwise, throughout the description above and the appended claims, the words “comprise,” “comprising,” and the like are to be construed in an inclusive sense as opposed to an exclusive or exhaustive sense; that is to say, in a sense of “including, but not limited to.” Words using the singular or plural number also include the plural or singular number, respectively. Additionally, the words “herein,” “hereunder,” “above,” “below,” and words of similar import refer to this application as a whole and not to any particular portions of this application. When the word “or” is used in reference to a list of two or more items, that word covers all of the following interpretations of the word: any of the items in the list, all of the items in the list, and any combination of the items in the list

The foregoing description, for purpose of explanation, has been described with reference to specific embodiments. However, the illustrative discussions above are not intended to be exhaustive or to limit the invention to the precise forms disclosed. Many modifications and variations are possible in view of the above descriptions. The embodiments were chosen and described in order to best explain the principles of the invention and its practical applications to thereby enable others skilled in the art to best utilize the invention and various embodiments with various modifications as are suited to the particular use contemplated. As such, the invention extends to all functionally equivalent structures, methods, and uses, such as are within the scope of the appended claims, and it is intended that the invention be limited only to the extent required by the applicable rules of law.

What is claimed:

1. A disposable, single-use medical connector access port cover, comprising:
a. a housing with a recess, the housing adapted to receive a port cover matrix and comprising a removal tab;
b. a port cover matrix comprised of an absorbent compressible material in which is disposed an antimicrobial agent; and
c. a removable seal.

2. An access port cover according to claim 1 packaged in foiled seal pouch.

3. An access port cover according to claim 1 that has been sterilized.

4. A method of reducing contamination of a valve surface of a needleless medical connector, comprising removably attaching an access port cover according to claim 1 to a needleless medical connector comprising a fluid valve such that the surface of the valve contacts the port cover matrix of the access port cover.
